(19) United States
(12) Patent Application Publication
(10) Pub. No.: US 2020/0086038 A1
(43) Pub. Date: Mar. 19, 2020

(54) CAP AND METHODS OF MAKING AND USING SAME

(71) Applicant: MATRONEÉ LLC, Shawnee, OK (US)

(72) Inventor: Joni Hawkins, Shawnee, OK (US)

(73) Assignee: MATRONEÉ LLC

(21) Appl. No.: 16/573,757

(22) Filed: Sep. 17, 2019

Related U.S. Application Data
(60) Provisional application No. 62/732,448, filed on Sep. 17, 2018.

(51) Int. Cl. A61M 5/14; A42B 1/00

(52) U.S. Cl. CPC A61M 5/1415; A42B 1/006; A61M 2240/00; A61M 2205/0216; A61M 2005/1416; A61M 2205/584

(57) ABSTRACT

The present disclosure is directed to a cap to be worn by an individual having a scalp intravenous tube so as to help in maintaining the body temperature of the individual. The cap includes a body, at least one opening and a fastener. The body has a plurality of panels. The at least one opening is formed between the plurality of panels of the body. The fastener is positioned on a portion of at least one of the plurality of panels of the body to connect the plurality of panels of the body so as to attach the body of the cap to a head of an individual.

### CROSS REFERENCE TO RELATED APPLICATIONS/INCORPORATION BY REFERENCE STATEMENT

The present patent application claims priority to U.S. Provisional Patent Application U.S. Ser. No. 62/732,448, filed on Sep. 17, 2018, the entire contents of which is hereby expressly incorporated herein by reference.

### FIELD OF THE INVENTION

The present disclosure relates generally to a cap for use on an individual, and more particularly, but not by way of limitation, to an improved cap for use on an individual to help maintain body temperature and for providing an opening for administering scalp intravenous therapy and methods of making and using the improved cap.

### BACKGROUND

Placing an IV line into a peripheral vein in a small child or infant can be a difficult task, for any reasons. Small children and infants have smaller peripheral veins, they may have more subcutaneous fat, they are prone to vasoconstriction, and they are much less likely to remain motionless and cooperative during a painful procedure than adults. Thus, at times, a scalp IV is necessary.

Babies are not as adjustable as adults to temperature changes. Babies can lose heat rapidly, as much as four times more quickly than adults. Premature and low birthweight babies usually have little body fat and may be too immature to regulate their own temperature. When babies are stressed by cold, they use energy and oxygen to generate warmth. If skin temperatures drop just one degree from the ideal temperature, a baby's oxygen can decrease by 10%. By keeping babies warm at optimal temperatures, they can conserve energy and build up energy reserves. This is especially important when babies are sick or premature. A baby is wrapped in a blanket and a cap is placed on his/her head since a baby can lose large amounts of heat through his/her head.

Placing an IV in the scalp of the infant makes it difficult for nurses and other hospital staff to maintain body temperature in newborns/infants in the hospital nursery or the Neonatal Intensive Care Unit.

To this end, a need exists for an improved cap that provides an opening for a scalp IV and for helping to maintain the body temperature of an infant. It is to such an improved cap that the present disclosure is directed.

### BRIEF DESCRIPTION OF THE SEVERAL VIEWS OF THE DRAWINGS

FIG. 1 is a perspective view of one embodiment of an improved cap positioned on an infant, the cap constructed in accordance with the present disclosure.

FIG. 2 is a perspective top view of the cap of FIG. 1.

FIG. 3 is a front view of another embodiment of an improved cap constructed in accordance with the present disclosure.

### DETAILED DESCRIPTION

Before explaining at least one embodiment of the inventive concept disclosed herein in detail, it is to be understood that the inventive concept is not limited in its application to the details of construction, experiments, exemplary data, and/or the arrangement of the components set forth in the following description, or illustrated in the drawings. The presently disclosed and claimed inventive concept is capable of other embodiments or of being practiced or carried out in various ways. Also, it is to be understood that the phraseology and terminology employed herein is for purpose of description only and should not be regarded as limiting in any way.

In the following detailed description of embodiments of the inventive concept, numerous specific details are set forth in order to provide a more thorough understanding of the inventive concept. However, it will be apparent to one of ordinary skill in the art that the inventive concept within the disclosure may be practiced without these specific details. In other instances, well-known features have not been described in detail to avoid unnecessarily complicating the instant disclosure.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or at least one and the singular also includes the plural unless it is obvious that it is meant otherwise.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

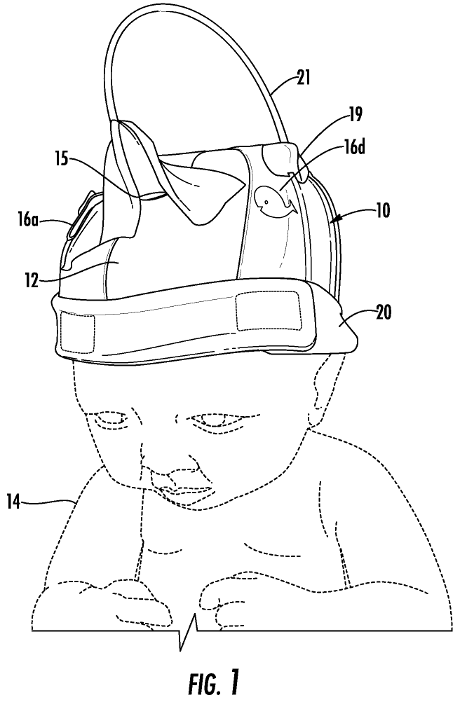

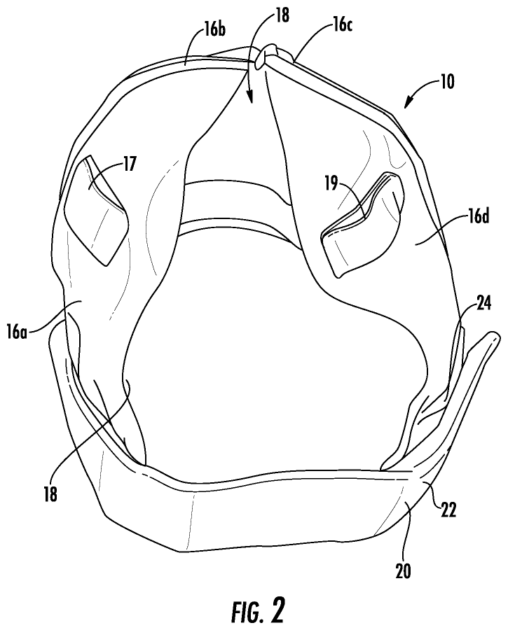

Referring now to the drawings, and more particularly to FIGS. 1-2, one embodiment of a cap 10 constructed in accordance with the present disclosure is shown positioned about a head 12 of an infant 14 having an IV 15. The cap 10 may be made from acrylic, cotton, fleece, wool or any known material for making a cap 10. Additionally, the cap 10 may be constructed from a washable and/or disposable material.

Broadly, the cap 10 includes a plurality of panels 16a-16d, at least one opening 18 and a fastener 20. Each panel 16 is shown as substantially triangular in shape, however, it should be understood by one of ordinary skill in the art that any shaped panel may be utilized in the cap 10, such as square, rectangular, circular, trapezoidal or the like or the panels may be one continuous single piece of material, so long as the cap 10 functions in accordance with the present disclosure. The cap 10 may employ bright primary colors and print fabric to lessen the startling impact of the scalp IV for parents and caregivers. Each panel 16 may be provided with the same or a different color(s) and/or design. Color coding indicating size to ensure proper fit of the cap and to ease the visual impact of the IV.

The plurality of panels 16 are joined along lateral edges of each panel to form the hat 10 and conform to the shape of the head 12 the infant 14. In one embodiment, no interior seams are provided to keep pressure and irritants away from the infant's delicate scalp. A hook and loop fastener 17 is provided on the panel 16a and a hoop and loop fastener 19 is provided on panel 16d for use in keeping the tubing 21 away from the opening 18 of the cap 10. It should be understood that any number of fasteners may be included on the plurality of panels 16 so long as the fasteners function in accordance with the present disclosure. In another embodiment, a cuff (not shown) may be included about a portion of the plurality of panels and constructed of an elastic material so as to help in further positioning the hat about the head of the infant.

In one embodiment, the opening 18 is formed by removing at least one of the plurality of panels. 16. The opening 18 allows for the cap to be positioned so that various locations for the scalp IV 15 may be utilized (including the superficial, temporal, frontal, occipital and pre- and post-auricular).

The fastener 20 is provided with a hook and loop type fastener strip 22 which is adapted to be connected to a corresponding hook and loop type fastener strip 24 secured to a portion of the plurality of panels 16 to permit the hat 10 to be adjustably fastened to the infant's head 12 upon mating the hook and loop type fastener strip 22 with the hook and loop type fastener strip 24, and yet permit the cap 10 to be easily removed from the infant's head 12 upon releasing the hook and loop type fastener strip 22 from the hook and loop type fastener strip 24. The hook and loop strips 22 and 24 located on each side of the opening 18 direct and restrain IV lines away from the face of the infant without additional tape and aid in nursing or feeding without fear of tugging or dislocating the IV. It will be appreciated by those of ordinary skill in the art that numerous types of fasteners are known in the art which would be suitable for use in the glove 10. It should be understood by one of ordinary skill in the art that any type of fastening device for the purpose of maintaining the panels of the hat 10 in place about the head of the infant may be utilized, such as but not limited to dips, buttons, two-sided tape, adhesives, and the like. It is further appreciated that instead of fasteners, lateral panels can be held in place in a “belt and loop” arrangement wherein the loop is formed by cutting a slit into an opposite lateral pan& for receiving the free end of the other lateral panel.

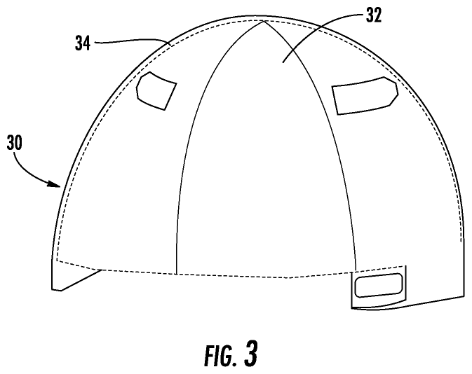

Referring to FIG. 3, shown therein, is another embodiment of an improved cap 30 substantially the same as the cap 10 except that a plurality of openings 32 and 34 (two panels removed) to allow additional access areas. It should be understood that any number of openings may be provided so long as the cap functions in accordance with the present disclosure.

Although the examples herein describe the use of the cap with an infant, it will be understood by one of ordinary skill in the art, that the cap may be sized and dimensioned to be used on any sized individual such as a child, teenager and/or adult. It should be understood that changes may be made in the operation and the setup of the embodiment.

From the above description, it is clear that the inventive concept(s) disclosed herein is well adapted to carry out the objects and to attain the advantages mentioned herein as well as those inherent in the inventive concept disclosed herein. While exemplary embodiments of the inventive concept disclosed herein have been described for purposes of this disclosure, it will be understood that numerous changes may be made which will readily suggest themselves to those skilled in the art and which are accomplished without departing from the scope of the inventive concept disclosed herein.

What is claimed is:

1. A cap, comprising:
a body having a plurality of panels;
at least one opening formed between the plurality of panels of the body; and
a fastener positioned on a portion of at least one of the plurality of panels of the body to connect the plurality of panels of the body so as to attach the body of the cap to a head of an individual.

2. The cap of claim 1 wherein each of the plurality of panels is substantially triangular.

3. The cap of claim 1 where each of the plurality of panels of the body is connected to one another along a lateral edge of each of the plurality of panels of the body.

4. The cap of claim 1 wherein at least one of the plurality of panels is removed to create the at least one opening.

5. The cap of claim 1 wherein the fastener is a hook and loop type material.

6. The cap of claim 1 further comprising:
a second fastener positioned on a portion of the plurality of panels for keeping a tube away from the at least one opening of the cap.

7. A cap, comprising:
a plurality of panels forming a body;
an opening positioned between the plurality of panels; and
a connecting member positioned on a portion of at least one of the plurality of panels to connect the plurality of panels to be attached to an individual's head.

8. The cap of claim 7 wherein each of the plurality of panels is substantially triangular.

9. The cap of claim 7 where each of the plurality of panels is connected to one another along a lateral edge of each of the plurality of panels.

10. The cap of claim 7 wherein at least one of the plurality of panels is removed to create the opening.

12. The cap of claim 7 wherein the connecting member is a hook and loop type material.

13. The cap of claim 7 further comprising:
a second connecting member positioned on a portion of the plurality of panels for keeping a tube away from the opening of the cap.

14. A method of attaching a cap to the head of an individual, the steps comprising:
providing a cap having an opening formed on a portion of a body of the cap;
positioning the cap on the head of an individual so that the opening is positioned over a scalp intravenous tube; and
attaching the body of the cap to the head of the individual to maintain a body temperature of the individual.

15. The method of claim 14, wherein the body of the cap, comprising:
a plurality of panels, wherein the opening is formed between the plurality of panels of the body; and
a fastener positioned on a portion of at least one of the plurality of panels of the body to connect the plurality of panels of the body so as to attach the body of the bap to a head of an individual.

16. The method of claim 15 wherein each of the plurality of panels is substantially triangular.

17. The method of claim 15 where each of the plurality of panels of the body is connected to one another along a lateral edge of each of the plurality of panels of the body.

18. The method of claim 15 wherein at least one of the plurality of panels is removed to create the opening.

19. The method of claim 15 wherein the fastener is a hook and loop type material.

20. The method of claim 15 further comprising:
a second fastener positioned on a portion of the plurality of panels for keeping a tube away from the opening of the cap.
